(19) United States
(12) Patent Application Publication
(10) Pub. No.: US 2013/0281789 A1
(43) Pub. Date: Oct. 24, 2013

(54) Eye Opening Device

(71) Applicants: William A. Stack, Superior, CO (US); Darrell K. Stack, Casper, WY (US)

(72) Inventors: William A. Stack, Superior, CO (US); Darrell K. Stack, Casper, WY (US)

(21) Appl. No.: 13/451,788

(22) Filed: Apr. 20, 2012

(51) Int. Cl. A61F 9/00; A61B 1/32

(52) U.S. Cl. 600/219

(57) ABSTRACT

A device comprises a first member comprising a tong portion, a pivot point, and a handle portion. A second member comprises a tong portion, a pivot point, and a handle portion, in which the first member and the second member are joined at the pivot points. A first contact member comprises a surface being configured for contacting a tissue portion. The first contact member is joined to a distal end of the first member with the surface generally facing away from a plane formed by the joined tong portions. A second contact member comprises a surface being configured for contacting a tissue portion. The second contact member is joined to a distal end of the second member with the surface generally facing away from the plane, in which a rotation of the first member and the second member about the pivot point is operable for separating the tissue portions.

### FEDERALLY SPONSORED RESEARCH OR DEVELOPMENT

Not applicable.

### REFERENCE TO SEQUENCE LISTING, A TABLE, OR A COMPUTER LISTING APPENDIX

Not applicable.

### COPYRIGHT NOTICE

A portion of the disclosure of this patent document contains material that is subject to copyright protection. The copyright owner has no objection to the facsimile reproduction by anyone of the patent document or patent disclosure as it appears in the Patent and Trademark Office, patent file or records, but otherwise reserves all copyright rights whatsoever.

### FIELD OF THE INVENTION

One or more embodiments of the invention generally relate to personal care items. More particularly, one or more embodiments of the invention relate to an eye opening device.

### BACKGROUND OF THE INVENTION

The following background information may present examples of specific aspects of the prior art (e.g., without limitation, approaches, facts, or common wisdom) that, while expected to be helpful to further educate the reader as to additional aspects of the prior art, is not to be construed as limiting the present invention, or any embodiments thereof, to anything stated or implied therein or inferred thereupon. It is believed that many individuals have trouble wearing contact lenses because they are not able to open their eyes wide enough to insert the contact lenses properly. Similarly, these individuals may also have trouble performing other tasks that typically require access to the eyeball such as, but not limited to, removing foreign objects from the eye, applying eye drops, flushing the eye, examining the eye, etc.

By way of educational background, an aspect of the prior art generally useful to be aware of is that the eyes may be opened wide using the fingers. However, some individuals may have small eyes that are difficult to open with the fingers. Furthermore, the fingers may get in the way or may carry dirt or germs that could fall into the eye.

In view of the foregoing, it is clear that these traditional techniques are not perfect and leave room for more optimal approaches.

### BRIEF DESCRIPTION OF THE DRAWINGS

The present invention is illustrated by way of example, and not by way of limitation, in the figures of the accompanying drawings and in which like reference numerals refer to similar elements and in which:

FIGS. 1A through 1C illustrate an exemplary eye opening device, in accordance with an embodiment of the present invention. FIG. 1A is a diagrammatic side view. FIG. 1B is a side perspective view of eye contacts, and FIG. 1C is an exploded view of the eye opening device;

FIG. 2 is an exploded view of an exemplary eye opening device, in accordance with an embodiment of the present invention;

FIG. 3 is a diagrammatic side view of an exemplary locking mechanism for an eye opening device, in accordance with an embodiment of the present invention; and

FIG. 4 is a diagrammatic top view of an exemplary contact of an eye opening device with a variable hinge, in accordance with an embodiment of the present invention.

Unless otherwise indicated illustrations in the figures are not necessarily drawn to scale.

### DETAILED DESCRIPTION OF SOME EMBODIMENTS

Embodiments of the present invention are best understood by reference to the detailed figures and description set forth herein.

Embodiments of the invention are discussed below with reference to the Figures. However, those skilled in the art will readily appreciate that the detailed description given herein with respect to these figures is for explanatory purposes as the invention extends beyond these limited embodiments. For example, it should be appreciated that those skilled in the art will, in light of the teachings of the present invention, recognize a multiplicity of alternate and suitable approaches, depending upon the needs of the particular application, to implement the functionality of any given detail described herein, beyond the particular implementation choices in the following embodiments described and shown. That is, there are numerous modifications and variations of the invention that are too numerous to be listed but that all fit within the scope of the invention. Also, singular words should be read as plural and vice versa and masculine as feminine and vice versa, where appropriate, and alternative embodiments do not necessarily imply that the two are mutually exclusive.

It is to be further understood that the present invention is not limited to the particular methodology, compounds, materials, manufacturing techniques, uses, and applications, described herein, as these may vary. It is also to be understood that the terminology used herein is used for the purpose of describing particular embodiments only, and is not intended to limit the scope of the present invention. It must be noted that as used herein and in the appended claims, the singular forms “a,” “an,” and “the” include the plural reference unless the context clearly dictates otherwise. Thus, for example, a reference to “an element” is a reference to one or more elements and includes equivalents thereof known to those skilled in the art. Similarly, for another example, a reference to “a step” or “a means” is a reference to one or more steps or means and may include sub-steps and subservient means. All conjunctions used are to be understood in the most inclusive sense possible. Thus, the word “or” should be understood as having the definition of a logical “or” rather than that of a logical “exclusive or” unless the context clearly necessitates otherwise. Structures described herein are to be understood also to refer to functional equivalents of such structures. Language that may be construed to express approximation should be so understood unless the context clearly dictates otherwise.

Unless defined otherwise, all technical and scientific terms used herein have the same meanings as commonly understood by one of ordinary skill in the art to which this invention belongs. Preferred methods, techniques, devices, and materials are described, although any methods, techniques, devices, or materials similar or equivalent to those described herein may be used in the practice or testing of the present invention. Structures described herein are to be understood also to refer to functional equivalents of such structures. The present invention will now be described in detail with reference to embodiments thereof as illustrated in the accompanying drawings.

From reading the present disclosure, other variations and modifications will be apparent to persons skilled in the art. Such variations and modifications may involve equivalent and other features which are already known in the art, and which may be used instead of or in addition to features already described herein.

Although Claims have been formulated in this Application to particular combinations of features, it should be understood that the scope of the disclosure of the present invention also includes any novel feature or any novel combination of features disclosed herein either explicitly or implicitly or any generalization thereof, whether or not it relates to the same invention as presently claimed in any Claim and whether or not it mitigates any or all of the same technical problems as does the present invention.

Features which are described in the context of separate embodiments may also be provided in combination in a single embodiment. Conversely, various features which are, for brevity, described in the context of a single embodiment, may also be provided separately or in any suitable subcombination. The Applicants hereby give notice that new Claims may be formulated to such features and/or combinations of such features during the prosecution of the present Application or of any further Application derived therefrom.

References to “one embodiment,” “an embodiment,” “example embodiment,” “various embodiments,” etc., may indicate that the embodiment(s) of the invention so described may include a particular feature, structure, or characteristic, but not every embodiment necessarily includes the particular feature, structure, or characteristic. Further, repeated use of the phrase “in one embodiment,” or “in an exemplary embodiment,” do not necessarily refer to the same embodiment, although they may.

As is well known to those skilled in the art many careful considerations and compromises typically must be made when designing for the optimal manufacture of a commercial implementation any system, and in particular, the embodiments of the present invention. A commercial implementation in accordance with the spirit and teachings of the present invention may configured according to the needs of the particular application, whereby any aspect(s), feature(s), function(s), result(s), component(s), approach(es), or step(s) of the teachings related to any described embodiment of the present invention may be suitably omitted, included, adapted, mixed and matched, or improved and/or optimized by those skilled in the art, using their average skills and known techniques, to achieve the desired implementation that addresses the needs of the particular application.

It is to be understood that any exact measurements/dimensions or particular construction materials indicated herein are solely provided as examples of suitable configurations and are not intended to be limiting in any way. Depending on the needs of the particular application, those skilled in the art will readily recognize, in light of the following teachings, a multiplicity of suitable alternative implementation details.

One embodiment of the present invention provides a device to assist in opening the eye. Some embodiments may be used for insertion of contact lenses, application of eye drops, eye examinations, removal of foreign objects, flushing the eye, performing eye surgery, etc. Many embodiments allow direct access to the eye ball through a scissor like action.

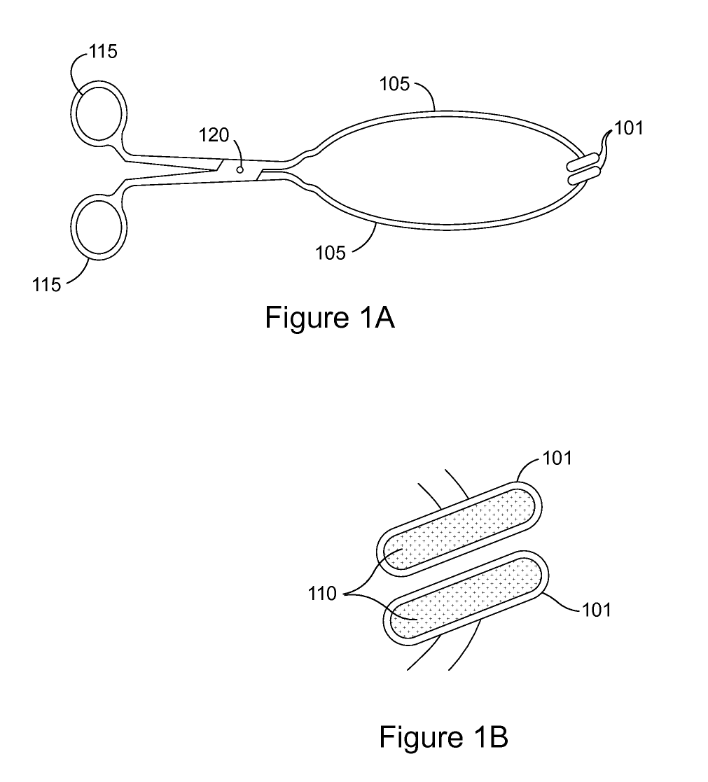

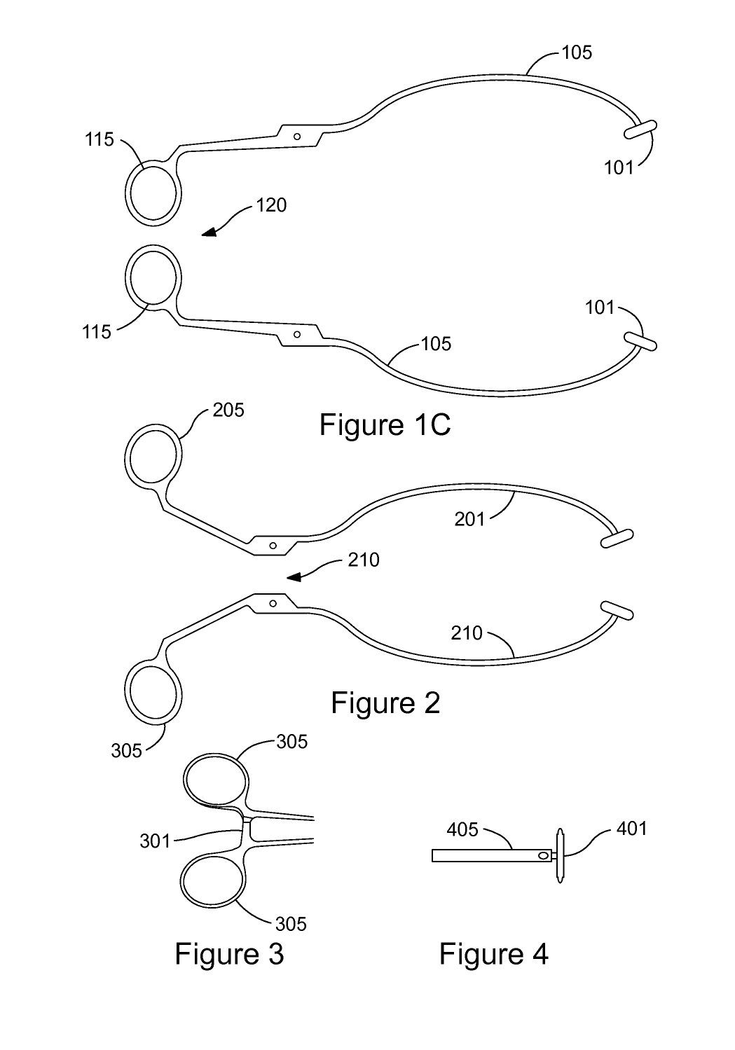

FIGS. 1A through 1C illustrate an exemplary eye opening device, in accordance with an embodiment of the present invention. FIG. 1A is a diagrammatic side view. FIG. 1B is a side perspective view of eye contacts 101, and FIG. 1C is an exploded view of the eye opening device. In the present embodiment, the device is constructed using a basic scissor action. Contacts 101 are attached to the ends of curved tongs 105 that allow access to the eye by extending above and below the eye. Contacts 101 are angled at approximately 30 degrees to generally allow for clear visual access in a mirror of the eye and physical access to the eye. It is contemplated that the contacts in some alternate embodiments may be angled at various different angles, including, without limitation, no angle. Other alternate embodiments may have contacts with adjustable angles. Referring to FIG. 1B, in the present embodiment, the surface of contacts 101 comprises a fine crisscross cut texture 110 to better adhere to the eyelid during use. Contacts in some alternate embodiments may be implemented with a multiplicity of suitable textures such as, but not limited to, ridged textures, knurled textures, smooth textures, etc.

In the present embodiment, loops 115 are located on the manipulation end of the device into which a user's fingers may be inserted to open and close the device around a connection pin 120. With the present configuration of tongs 105 and loops 120, the device is opened by moving loops 120 apart to open the manipulation end of the device and closed by moving loops 120 together to close the manipulation end of the device. Some alternate embodiments may be configured differently, including, without limitation, the embodiment illustrated by example in FIG. 2. In the present embodiment, the device is made of surgical grade stainless steel as the use of this material may allow for medical use of the device and generally enables the device to be sterilized in an autoclave. Those skilled in the art will readily recognize, in light of and in accordance with the teachings of the present invention, that eye opening devices in some alternate embodiments may be made of a multiplicity of suitable materials such as, but not limited to, various different metals or plastics.

In typical use of the present embodiment, a user places his thumb and forefinger into loops 115 and places contacts 101 on his closed eyelid. Contacts 101 are generally placed as close as possible to the opening of the eyelid with one contact 101 on each side of the opening. The user then slowly opens the device to open the eyelid as far as is comfortably possible, while applying slight pressure towards the eye. Once open, clear access is provided to the eye for applications such as, but not limited to, inserting contact lenses, looking for foreign matter, putting in drops, examining the eye, flushing the eye, surgery, etc. When finished, the user closes the eye opening device and pulls the device away from the eye.

By using an eye opening device according to the present embodiment, the user is typically able to open the eye wider than conventional methods of opening the eye using the fingers to generally provide easier access to the surface of the eye as the fingers are not in the way. Also, contacts 101 may cause the lips of the eyelid to curl away from the eyeball, creating an even wider opening. Furthermore, some embodiments may be sterilized as opposed to fingers that may carry germs or other undesirable matter. It is believed that many embodiments may be more comfortable than using fingers and may cause less anxiety. If used properly, the eye opening device may be more stable compared to holding the eyelid open with fingers. The device also may provide others, including, without limitation, doctors, nurses, parents, etc., sterile access to eyes to insert drops, remove objects, examine the eyes, for surgery or surgery preparation, etc. In the present embodiment, the eye opening device is a simple mechanical device that can be used in virtually any environment, including, without limitation, homes, offices, hospitals, ambulances, veterinarians' offices, schools, cars, at the beach, while hiking, in travel kits, etc. An alternate use for the eye opening device according to the present embodiment is to apply pressure to keep another part of the body open, such as, but not limited to, a small cut while looking for a sliver in the cut or gently spreading a nostril open while looking for something a child may have put up their nose. Furthermore, some alternate embodiments may be implemented to hold the eyelids open on animals for the same purposes previously listed.

FIG. 2 is an exploded view of an exemplary eye opening device, in accordance with an embodiment of the present invention. In the present embodiment, curved tongs 201 and loops 205 are configured so that the device operates around a connection pin 210 with a close-to-open action as opposed to the open-to-open action illustrated by way of example in the foregoing embodiment.

FIG. 3 is a diagrammatic side view of an exemplary locking mechanism 301 for an eye opening device, in accordance with an embodiment of the present invention. In the present embodiment, locking device 301 comprises interlocking tabs on loops 305. Locking device 301 may be used on a device with a close-to-open action to hold the device and the eyelid open without continually applying pressure. Alternatively, locking mechanism 301 may be used to hold a device with an open-to-open action in a closed position when not in use. Those skilled in the art will readily recognize, in light of and in accordance with the teachings of the present invention, that a multiplicity of suitable locking means may be used in some alternate embodiments, such as, but not limited to, sleeves or bands around the tongs, means for tightening the tongs at the connection pin, locking pins, etc.

FIG. 4 is a diagrammatic top view of an exemplary contact 401 of an eye opening device with a variable hinge 405, in accordance with an embodiment of the present invention. In the present embodiment, hinge 405 enables a user to choose the angle of use of contact 401. Hinge 405 may also enable contact 401 to move during use, which may make contact 401 able to adjust to the shape of the user's eye for added comfort.

It is contemplated that some alternate embodiments may comprise a multiplicity of suitable additional features such as, but not limited to, tongs and contacts of various different sizes, interchangeable and/replaceable contacts, padding on the loops of the handle, ergonomically shaped loops, lights, etc. Furthermore, some alternate embodiments may include, without limitation, various different accessories such as, but not limited to, holsters, etc.

All the features disclosed in this specification, including any accompanying abstract and drawings, may be replaced by alternative features serving the same, equivalent or similar purpose, unless expressly stated otherwise. Thus, unless expressly stated otherwise, each feature disclosed is one example only of a generic series of equivalent or similar features.

Having fully described at least one embodiment of the present invention, other equivalent or alternative methods of providing an eye opening device according to the present invention will be apparent to those skilled in the art. The invention has been described above by way of illustration, and the specific embodiments disclosed are not intended to limit the invention to the particular forms disclosed. For example, the particular implementation of the device may vary depending upon the particular type of actuation means used. The devices described in the foregoing were directed to scissor like implementations; however, similar techniques are to provide eye opening devices that open and close using various different means such as, but not limited to, accordion type mechanisms, spring loaded mechanisms, sliding mechanisms, etc. Non-scissor implementations of the present invention are contemplated as within the scope of the present invention. The invention is thus to cover all modifications, equivalents, and alternatives falling within the spirit and scope of the following claims.

Claim elements and steps herein may have been numbered and/or lettered solely as an aid in readability and understanding. Any such numbering and lettering in itself is not intended to and should not be taken to indicate the ordering of elements and/or steps in the claims.

What is claimed is:

1. A device comprising:
a first member comprising a first tong portion having a first distal end and a first proximal end, a pivot point disposed proximate said first proximal end, and a first handle portion extending beyond said pivot point;
a second member comprising a second tong portion having a second distal end and a second proximal end, a pivot point disposed proximate said second proximal end, and a second handle portion extending beyond said pivot point, in which said first member and said second member are configured for rotatable joining at said pivot points with said first distal end opposing said second distal end;
a first contact member comprising a first surface being configured for contacting a first biological tissue portion, said first contact member being joined to said first distal end with said first surface generally facing away from a plane formed by said joined first tong portion and said second tong portion; and
a second contact member comprising a second surface being configured for contacting a second biological tissue portion, said second contact member being joined to said second distal end with said second surface generally facing away from the plane, in which a rotation of said first member and said second member about said pivot point is operable for separating the first biological tissue portion from the second biological tissue portion.

2. The device as recited in claim 1, in which said first tong portion and said second tong portion each further comprise a curved shape.

3. The device as recited in claim 2, in which said joined first tong portion and said second tong portion generally form a loop.

4. The device as recited in claim 1, in which said first handle portion and said second handle portion each further comprise a loop structure being configured for at least an insertion of a finger for operating said rotation.

5. The device as recited in claim 1, in which said first surface and said second surface each further comprise a texture being configured for adhering to the biological tissue.

6. The device as recited in claim 1, further comprising a locking mechanism being configured for retaining the device at a selected rotational position.

7. The device as recited in claim 1, in which said first surface and said second surface each further comprise a generally rectangular shape.

8. The device as recited in claim 1, further comprising a pin member in which said first member and said second member are further configured for rotatable joining about said pin member.

9. The device as recited in claim 1, further comprising a first hinge mechanism for joining said first contact member to said first distal end, and a second hinge mechanism for joining said second contact member to said second distal end.

10. The device as recited in claim 1, in which the device comprises a material suitable for autoclaving.

11. The device as recited in claim 10, in which said material comprises surgical grade stainless steel.

12. The device as recited in claim 1, in which the first biological tissue portion comprises an upper eyelid and the second biological tissue portion comprises a lower eyelid.

13. An eye opening device comprising:
first means being configured to be operable for rotating about a pivot point by least an insertion of a finger;
second means being configured to be operable for rotating about said pivot point by least an insertion of a finger;
first contact means being configured for contacting an eyelid, said first contact means being joined to a distal end of said first rotating means with an eyelid contacting surface generally facing away from a plane formed by said first rotating means and said second rotating means; and
second contact means being configured for contacting an eyelid, said second contact means being joined to a distal end of said second rotating means with an eyelid contacting surface generally facing away from said plane, in which a rotation of said first rotating means and said second rotating means about said pivot point is operable for separating the eyelids.

14. The eye opening device as recited in claim 13, further comprising means being configured for retaining the device at a selected rotational position.

15. The eye opening device as recited in claim 13, further comprising means for adjustably joining said first contact means to said first rotating means, and means for adjustably joining said second contact means to said second rotating means.

16. An eye opening device comprising:
a first member comprising a first tong portion having a first distal end, a first proximal end and a curved shape, a pivot point disposed proximate said first proximal end, and a first handle portion extending beyond said pivot point, said first handle portion further comprising a loop structure being configured for at least an insertion of a finger;
a second member comprising a second tong portion having a second distal end, a second proximal end and a curved shape, a pivot point disposed proximate said second proximal end, and a second handle portion extending beyond said pivot point, said second handle portion further comprising a loop structure being configured for at least an insertion of a finger, in which said first member and said second member are configured for rotatable joining at said pivot points with said first distal end opposing said second distal end, and said first tong portion and said second tong portion generally forming a loop;
a first contact member comprising a first surface being configured for contacting an eyelid, said first surface further comprising a generally rectangular shape and a texture being configured for adhering to the eyelid, said first contact member being joined to said first distal end with said first surface generally facing away from a plane formed by said joined first tong portion and said second tong portion; and
a second contact member comprising a second surface being configured for contacting an eyelid, said second surface further comprising a generally rectangular shape and a texture being configured for adhering to the eyelid, said second contact member being joined to said second distal end with said second surface generally facing away from the plane, in which a rotation of said first member and said second member about said pivot point is operable for separating the eyelids.

17. The eye opening device as recited in claim 16, further comprising a locking mechanism being configured for retaining the device at a selected rotational position.

18. The eye opening device as recited in claim 16, further comprising a pin member in which said first member and said second member are further configured for rotatable joining about said pin member.

19. The eye opening device as recited in claim 16, further comprising a first hinge mechanism for joining said first contact member to said first distal end, and a second hinge mechanism for joining said second contact member to said second distal end.

20. The eye opening device as recited in claim 16, in which the device comprises a material suitable for autoclaving.
